(19) United States
(12) Patent Application Publication
(10) Pub. No.: US 2015/0190650 A1
(43) Pub. Date: Jul. 9, 2015

(54) FLEXIBLE EYE MASK

(71) Applicant: Innovate Photonics Limited, Swansea (GB)

(72) Inventor: David Simon George, Swansea (GB)

(73) Assignee: Innovate Photonics Limited, Swansea (GB)

(21) Appl. No.: 14/286,290

(22) Filed: May 23, 2014

(30) Foreign Application Priority Data
Jan. 6, 2014 (GB) 1400127.5

(51) Int. Cl. A61N 5/06

(52) U.S. Cl. CPC A61N 5/0618; A61N 2005/0626; A61N 2005/0648; A61N 2005/0652

(57) ABSTRACT

A flexible eye mask for inducing brainwave in a brain of a wearer including a flexible substrate conformable with the contours of a face of the wearer, a plurality of light generation devices disposed upon the flexible substrate, a controller for controlling operation of the plurality of light generation devices and a power source for powering the plurality of light generation devices.

### CROSS-REFERENCE TO RELATED APPLICATIONS

This patent application claims the benefit under 35 U.S.C. §119 of United Kingdom Patent Application No. 1400127.5, filed Jan. 6, 2014, which application is incorporated herein by reference.

### FIELD OF THE INVENTION

The present invention relates to a flexible eye mask for inducing brainwaves in the brain of a wearer of the mask.

### BACKGROUND OF THE INVENTION

The brain consists of millions of cells called neurons which communicate by sending electrical signals. The combined electrical activity of all the neurons is found to create a periodic electrical signal of the brain, commonly known as a brainwave. Brainwaves can be broadly separated into five categories according to the frequency of the electrical signal. These include gamma, beta, alpha, theta and delta brainwaves, with gamma representing the highest frequency range of >27 Hz and delta representing the lowest frequency range of <3 Hz. It has been shown that the type of brainwave occurring in an individual is representative of the activity of the individual and/or their mental state. For example, gamma waves are closely associated with an alert state, in which an individual may be processing information or forming ideas, whereas delta waves are more closely associated with deep, dreamless sleep.

Researchers have found that brainwaves can be stimulated to change a person's mental state, and this in turn can help with a variety of mental issues. This stimulation is typically performed by placing an individual in front of a light that is arranged to flash on and off. The frequency of the brainwaves are found to synchronise with the frequency of the flashing light such that the induced brainwave can be selected by selecting the appropriate frequency for the flashing light.

It is known to provide spectacles and goggles with light emitting diodes which flash on and off at a desired frequency to induce the desired brainwave. However, these spectacles and goggles are often cumbersome devices with rigid frames which are uncomfortable for the wearer, and which typically require the wearer to remain in a particular position to prevent the devices from dislocating from their desired position.

We have now devised a flexible eye mask for inducing brainwaves.

### BRIEF SUMMARY OF THE INVENTION

In accordance with the present invention, there is provided a flexible eye mask for inducing brainwaves in the brain of a wearer, the mask comprising a flexible substrate conformable with the contours of the face of the wearer, the mask further comprising a plurality of light generation devices disposed upon the substrate, a controller for controlling operation of the light generation devices and a power source for powering the light generation devices.

In an embodiment, the controller is arranged to selectively power the light generation devices to cause a synchronous flash of light to be generated from each device. The selective powering of the devices is created by a train of power pulses formed by the controller. The controller is further arranged to vary the frequency of the power pulses to match the frequency of the desired brainwave type.

In an embodiment, the light generation devices comprise light emitting diodes. The light emitting diodes are preferably arranged to generate light in the wavelength range of 400-700 nm.

In an embodiment, the substrate comprises a printed circuit board upon which the light generation devices, power source and controller are mounted. The printed circuit board may be formed of a flexible plastics material.

In an embodiment, the mask further comprises a flexible cover which is arranged to cover the substrate, controller and power source. Preferably, the cover is formed of a fabric.

In an embodiment, the cover comprises a plurality of apertures formed therein, which are alignable with the light generation devices on the substrate.

In an embodiment, the mask further comprises means, such as an elasticated strap, for securing the mask upon the face of the wearer.

These and other objects and advantages of the present invention will be readily appreciable from the following description of preferred embodiments of the invention and from the accompanying drawings and claims.

### BRIEF DESCRIPTION OF THE DRAWINGS

The nature and mode of operation of the present invention will now be more fully described in the following detailed description of the invention taken with the accompanying drawing figures, in which:

FIG. 1a is a front view of a flexible eye mask according to an embodiment of the present invention;

FIG. 1b is a rear view of the mask illustrated in FIG. 1a;

FIG. 2 is an exploded view of the mask illustrated in FIGS. 1a and 1b; and,

FIG. 3 is a schematic illustration of the mask during use.

### DETAILED DESCRIPTION OF THE INVENTION

At the outset, it should be appreciated that like drawing numbers on different drawing views identify identical, or functionally similar, structural elements of the invention. While the present invention is described with respect to what is presently considered to be the preferred aspects, it is to be understood that the invention as claimed is not limited to the disclosed aspects.

Furthermore, it is understood that this invention is not limited to the particular methodology, materials and modifications described and as such may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs. Although any methods, devices or materials similar or equivalent to those described herein can be used in the practice or testing of the invention, the preferred methods, devices, and materials are now described.

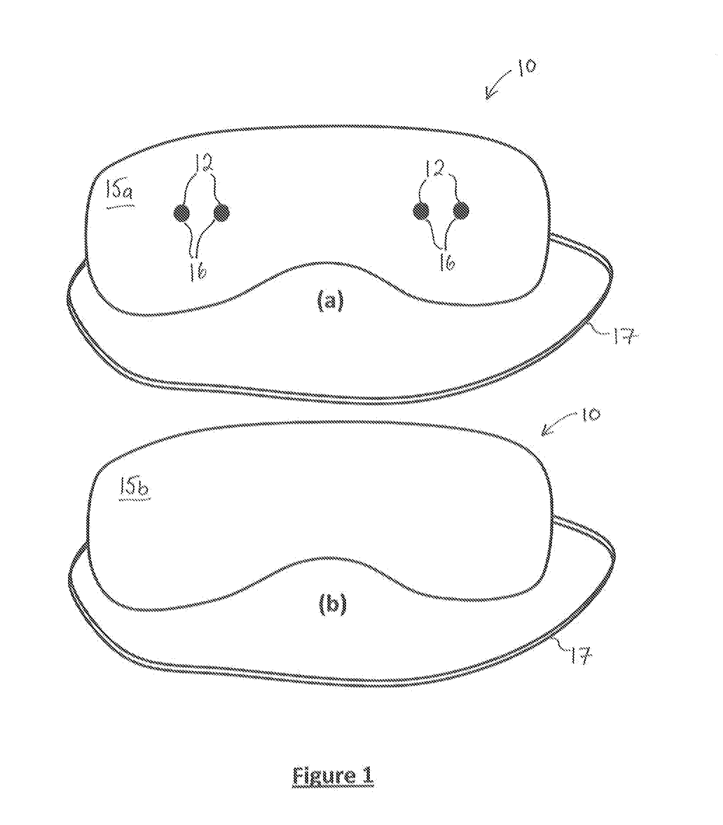

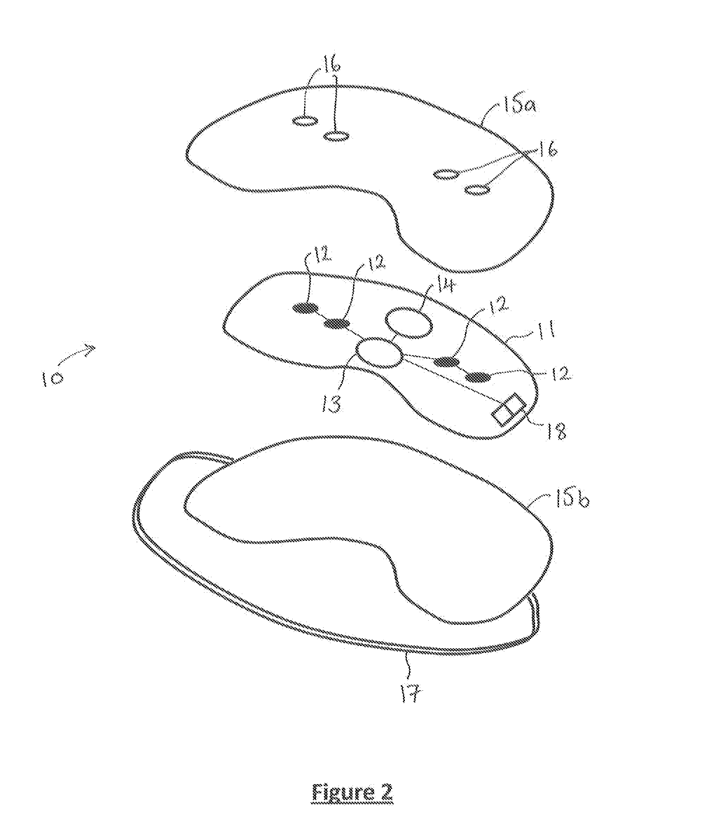

Referring to FIGS. 1 and 2 of the drawings, there is illustrated a flexible eye mask 10 according to an embodiment of the present invention for inducing brainwaves in the brain of a wearer 100. The mask 10 comprises a flexible substrate, such as a printed circuit board 11, which may be formed of a plastics material and which is conformable with the contours of the wearer, namely the eye-region of the wearers' face. The mask 10 further comprises a plurality of light generation devices 12, such as light emitting diodes (LEDs) disposed upon the substrate. In the illustrated embodiment, the mask 10 comprises tour LEDs 12 which are linearly aligned but grouped into pairs, with each pair being arranged to extend substantially adjacent an eye of a wearer. However, the skilled reader will recognise that different numbers of LEDs 12 may be used and configured in a different arrangement.

The mask 10 further comprises a controller 13 disposed upon the substrate 11, which is communicatively coupled with each of the LEDs 12. The controller 13 is arranged to control operation of the LEDs 12 by controlling the delivery of power to the LEDs 12 from a power source 14, such as a battery disposed upon the substrate 11. In an embodiment, the controller 13 is arranged to pulse the power to the LEDs 12 to provide for a synchronous flashing of light from the LEDs 12. The frequency of the flashing is determined by the frequency of the pulses of the train of pulses communicated from the controller 13 and so by varying the frequency of the pulses, the frequency of the flashing can be directly controlled.

In an embodiment, the flexible eye mask 10 further comprises a flexible cover 15 having a front and rear portion 15a, 15b which are arranged to encapsulate the substrate 11, controller 13 and power source 14. The cover 15 may be formed of a fabric material having a foam lining (not shown) for example, to improve comfort for the wearer 100 during use and also to protect the LEDs 12, controller 13 and power source 14 disposed upon the substrate 11. The front cover 15a, which in use is arranged to face the eye-region of the wearer, comprises a plurality of apertures 16 (four in the illustrated embodiment) formed therein which are located at positions corresponding to the position of the LEDs 12 on the substrate 11. The apertures 16 enable the flashes of light generated by the LEDs 12 to pass out from the cover 15, onto the eyes of the wearer 100 to facilitate the induction of brainwaves in the brain of the wearer 100.

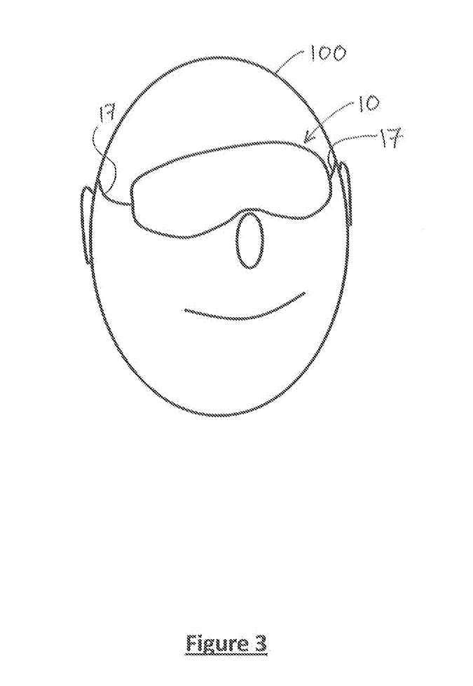

Referring to FIG. 3 of the drawings, in use the mask 10 is placed over the eye region of the wearer 100 such that the front cover 15a and thus LEDs 12 extend adjacent the wearers' eyes (not shown). The mask 10 can then be secured in place by placing a securing strap, such as an elasticated strap 17 which is coupled to opposite side edges of the mask 10, around the head of the wearer 100.

Once the mask 10 has been secured in place, the mask 10 may then be activated to initiate the flashing of the LEDs 12, by pressing a master switch 18 located on the substrate 11. The LEDs 12 are arranged to generate light in the wavelength range 400-700 nm, since this is the visible, namely receptive wavelength range for human eyes and thus which can provide a responsive reaction in the brain of the wearer.

The activation of the mask 10 causes the controller 13 to initially generate power pulses at a frequency of approximately 12 Hz. This frequency is subsequently reduced by the controller 13 over a pre-defined time period, such as 35 minutes, to a frequency of approximately 1 Hz. This frequency reduction is arranged to reduce the brain activity from that associated with the wearer 100 being in an awake state to a deep sleep state.

Therefore, it is evident that the flexible nature of the substrate 11 and cover 15 enable the mask 10 to be used comfortably while laying in bed, and can thus be used to alleviate the symptoms of insomnia.

Thus, it is seen that the objects of the present invention are efficiently obtained, although modifications and changes to the invention should be readily apparent to those having ordinary skill in the art, which modifications are intended to be within the spirit and scope of the invention as claimed. It also is understood that the foregoing description is illustrative of the present invention and should not be considered as limiting. Therefore, other embodiments of the present invention are possible without departing from the spirit and scope of the present invention.

What I claim is:

1. A flexible eye mask for inducing brainwaves in a brain of a wearer, the mask comprising:
a flexible substrate conformable with contours of a face of the wearer;
a plurality of light generation devices disposed upon the flexible substrate;
a controller for controlling operation of the plurality of light generation devices; and,
a power source for powering the plurality of light generation devices.

2. The flexible eye mask according to claim 1, wherein the controller is arranged to selectively power the plurality of light generation devices to cause a synchronous flash of light to be generated from each of the plurality of light generation devices.

3. The flexible eye mask according to claim 2, wherein the selective powering of the plurality of light generation devices is created by a train of power pulses formed by the controller.

4. The flexible eye mask according to claim 3, wherein the controller is arranged to vary a frequency of the train of power pulses to match a frequency of a desired type of brainwave.

5. The flexible eye mask according to claim 1, wherein the plurality of light generation devices comprise light emitting diodes.

6. The flexible eye mask according to claim 5, wherein the light emitting diodes are arranged to generate light in a wavelength range of 400-700 nm.

7. The flexible eye mask according to claim 1, wherein the flexible substrate comprises a printed circuit board upon which the plurality of light generation devices, the power source and the controller are mounted.

8. The flexible eye mask according to claim 1 further comprising:
a flexible cover which is arranged to cover the flexible substrate and power source.

9. The flexible eye mask according to claim 8, wherein the cover is formed of a fabric.

10. The flexible eye mask according to claim 8, wherein the cover comprises a plurality of apertures formed therein, which are alignable with the plurality of light generation devices on the flexible substrate.

11. The flexible eye mask according to claim 1 further comprising:
means for securing the mask upon the face of the wearer.
